(19) United States
(12) Patent Application Publication
(10) Pub. No.: US 2008/0194519 A1
(43) Pub. Date: Aug. 14, 2008

(54) TOPIRAMATE COMPOSITIONS AND METHODS FOR THEIR USE

(75) Inventor: James C. Cloyd, Edina, MN (US)

Correspondence Address: VIKSNINS HARRIS & PADYS PLLP, P.O. BOX 111098, ST. PAUL, MN, 55111-1098, US

(73) Assignee: REGENTS OF THE UNIVERSITY OF MINNESOTA, St. Paul, MN (US)

(21) Appl. No.: 11/855,642

(22) Filed: Sep. 14, 2007

Related U.S. Application Data
(60) Provisional application No. 60/844,875, filed on Sep. 15, 2006.

(51) Int. Cl. A61K 31/724; A61P 25/08; A61P 25/16

(52) U.S. Cl. 514/58

(57) ABSTRACT

The invention provides compositions that include topiramate and a cyclodextrin and methods for their use.

### RELATED APPLICATION(S)

This patent document claims the benefit of priority of U.S. application Ser. No. 60/844,875, filed Sep. 15, 2006, which application is herein incorporated by reference.

### STATEMENT OF GOVERNMENT SUPPORT

Work related to this patent document was funded by the U.S. government (NIH Grant NS-16308-26). The government may have certain rights in this patent document.

### BACKGROUND

Topiramate (2,3:4,5-Di-O-isopropylidene-β-D-fructopyranosesulfamate) is a well-known compound that is an anticonvulsant drug used to treat epilepsy in both children and adults. It is also approved for the treatment of migraines. However, while oral formulations of topiramate are available, injectable (e.g., intravenous (IV) or intramuscular (IM)) formulations of topiramate are needed.

There is also a need for compositions and methods for providing neuroprotection and for treating neonatal seizures.

### SUMMARY OF CERTAIN EMBODIMENTS OF THE INVENTION

As described herein, compositions suitable for injectable administration that include topiramate and a cyclodextrin have been developed. These injectable compositions are useful, e.g., for treating patient populations for which oral compositions of topiramate are not appropriate. For example, oral compositions of topiramate may not be appropriate because a patient may be too young, unable to swallow, undergoing GI surgery, incapacitated, or have a disorder that blocks absorption. Further, injectable compositions of topiramate would be useful for treating conditions where patients need to rapidly attain an increased concentration of topiramate. These injectable compositions also provide a more controlled dosing than do oral compositions.

Accordingly, certain embodiments of the present invention provide compositions comprising topiramate, or a salt thereof, and compound of Formula I:

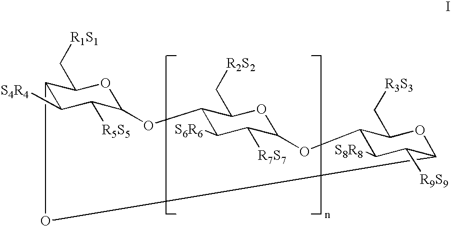

wherein:
n is 4, 5 or 6;
R₁, R₂, R₃, R₄, R₅, R₆, R₇, R₈ and R₉ are each, independently, —O— or a —O—(C₂-C₆ alkylene)-SO₃⁻ group, wherein at least one of R₁ and R₂ is independently a —O—(C₂-C₆ alkylene)-SO₃⁻ group; and
S₁, S₂, S₃, S₄, S₅, S₆, S₇, S₈ and S₉ are each, independently, H or a pharmaceutically acceptable cation.

Certain embodiments of the present invention provide compositions prepared by combining topiramate, or a salt thereof, and a compound of formula I:

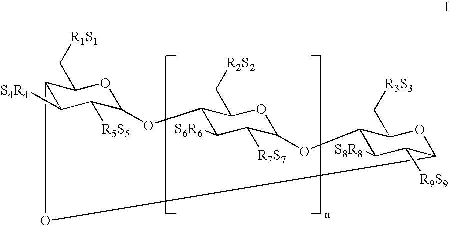

wherein:
n is 4, 5 or 6;
R₁, R₂, R₃, R₄, R₅, R₆, R₇, R₈ and R₉ are each, independently, —O— or a —O—(C₂-C₆ alkylene)-SO₃⁻ group, wherein at least one of R₁ and R₂ is independently a —O—(C₂-C₆ alkylene)-SO₃⁻ group; and
S₁, S₂, S₃, S₄, S₅, S₆, S₇, S₈ and S₉ are each, independently, H or a pharmaceutically acceptable cation.

In some embodiments of the invention, at least one of R₁ and R₂ is independently a —O—(C₂-C₆ alkylene)-SO₃⁻ group that is a —O—(CH₂)ₘSO₃⁻ group, wherein m is 2 to 6.

In some embodiments of the invention, the pharmaceutically acceptable cation is H, an alkali metal, an alkaline earth metal, an ammonium ion, or an amine cation.

Certain embodiments of the present invention provide compositions comprising topiramate, or a salt thereof, and a cyclodextrin such as a compound of Formula III:

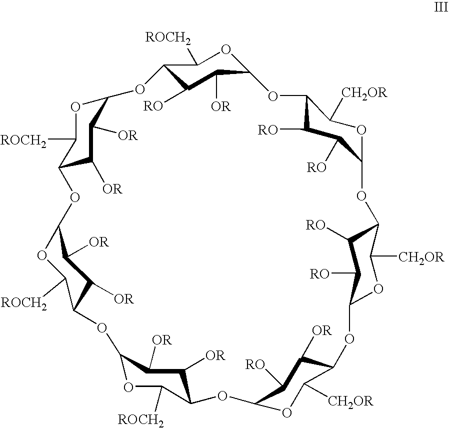

wherein R═(H)₂₁₋ₓ or (—(CH₂)₄—SO₃Na)ₓ. In some embodiments of the invention, x=6.0-7.1.

Certain embodiments of the present invention provide compositions prepared by combining topiramate, or a salt thereof, and a cyclodextrin such as a compound of Formula III:

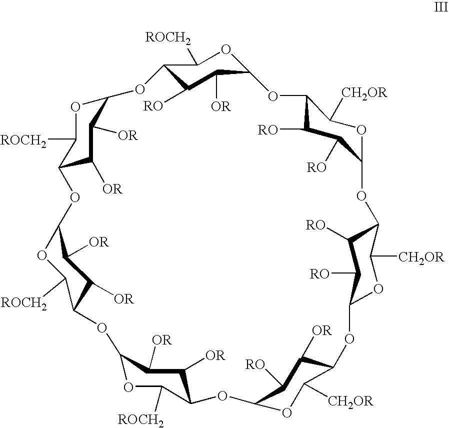

wherein R═(H)₂₁₋ₓ or (—(CH₂)₄—SO₃Na)ₓ. In some embodiments of the invention, x=6.0-7.1.

In some embodiments of the invention, the composition of the invention further comprises an additional therapeutic agent.

In some embodiments of the invention, the composition further comprises an additional pharmaceutically-acceptable carrier.

In some embodiments of the invention, the composition is suitable for injectable administration to a patient. In some embodiments of the invention, the composition is suitable for intravenous or intramuscular administration to a patient.

Certain embodiments of the present invention provide methods for delivering topiramate to a patient, comprising administering a composition of the invention to the patient. In some embodiments of the invention, the patient is a patient in need of treatment with topiramate. In some embodiments of the invention, the composition is administered intravenously to the patient.

Certain embodiments of the present invention provide methods for treating a patient who has or is at risk for developing a condition amenable to treatment with topiramate comprising administering an effective amount of a composition of the invention (e.g., intravenously) to the patient so as to treat the condition.

In some embodiments of the invention, the condition is selected from epilepsy, status epilepticus, refractory status epilepticus, gambling addiction, migraines, substance dependence, alcoholism, cocaine dependence, nicotine dependence, metabolic syndrome X, diabetes mellitus, type 2, vomiting, obsessive-compulsive disorder, refractory generalized social phobia, Tourette syndrome, levodopa-induced dyskinesia in Parkinson's Disease, refractory POS, Prader-Willi syndrome, multiple sclerosis, Lennox-Gastaut syndrome, bipolar disorder, obesity, post traumatic stress disorder, cluster headaches, severe headaches, and conditions caused by exposure to a chemical warfare nerve agents such as sarin.

Certain embodiments of the present invention provide methods for providing neuroprotection in a patient, comprising administering an effective amount of a composition of the invention (e.g., intravenously) to the patient. In some embodiments of the invention, the neuroprotection is needed during surgery.

Certain embodiments of the present invention provide methods for treating anoxia in a patient, comprising administering an effective amount of a composition of the invention (e.g., intravenously) to the patient.

Certain embodiments of the present invention provide methods for treating seizures in a patient, comprising administering an effective amount of a composition of the invention (e.g., intravenously) to the patient.

Certain embodiments of the present invention provide methods for loading a patient to attain an effective topiramate concentration, comprising administering an effective amount of a composition of the invention (e.g., intravenously) to the patient.

In some embodiments of the invention, oral topiramate therapy for the patient has been interrupted.

In some embodiments of the invention, the patient is a neonatal patient. In some embodiments of the invention, the patient is a pediatric patient. In some embodiments of the invention, the patient is an adult patient. In some embodiments of the invention, the patient is a geriatric patient.

Certain embodiments of the present invention provide compositions of the invention for use in medical treatment or diagnosis.

Certain embodiments of the present invention provide use of topiramate, or a salt thereof, and compound of Formula I:

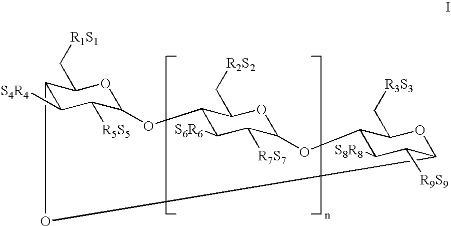

wherein:
n is 4, 5 or 6;
R₁, R₂, R₃, R₄, R₅, R₆, R₇, R₈ and R₉ are each, independently, —O— or a —O—(C₂-C₆ alkylene)-SO₃⁻ group, wherein at least one of R₁ and R₂ is independently a —O—(C₂-C₆ alkylene)-SO₃⁻ group; and
   S₁, S₂, S₃, S₄, S₅, S₆, S₇, S₈ and S₉ are each, independently, a pharmaceutically acceptable cation, to prepare a medicament useful for treating a condition amenable to treatment with topiramate in an animal.

Certain embodiments of the present invention provide the use of topiramate, or a salt thereof, and a cyclodextrin such as a compound of Formula III:

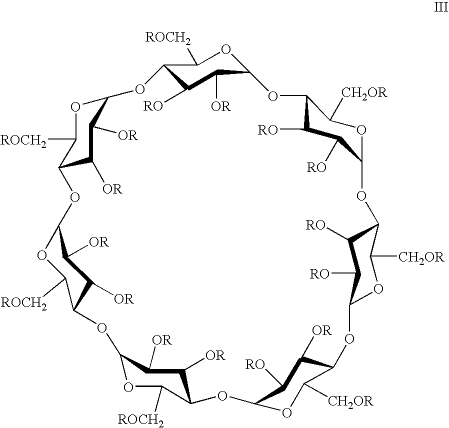

wherein R═(H)₂₁₋ₓ or (—(CH₂)₄—SO₃Na)ₓ, to prepare a medicament useful for treating a condition amenable to treatment with topiramate in an animal. In some embodiments of the invention, on average, x=6.0-7.1.

Certain embodiments of the present invention provide the use of a composition of the invention to prepare a medicament useful for treating a condition amenable to treatment with topiramate in an animal.

In some embodiments of the invention, the medicament is suitable for injectable (e.g., intravenous) administration to a patient.

### BRIEF DESCRIPTION OF THE FIGURES

FIG. 1 depicts a chromatogram from injection of a topiramate standard.

FIG. 2 depicts a chromatogram from injection of a topiramate standard.

FIG. 3 depicts results of a solubility study and demonstrates that topiramate is well solubilized by the cyclodextrin CAPTISOL® in water.

FIG. 4 depicts results of a solubility study and demonstrates that topiramate is well solubilized by the cyclodextrin CAPTISOL® in water.

FIG. 5 depicts a chromatogram for the analysis of the solubility sample using 40% w/v the cyclodextrin CAPTISOL®.

### DETAILED DESCRIPTION

Compositions for injectable (e.g., IV) administration of topiramate and a cyclodextrin have been developed.

In certain embodiments, the composition of the invention comprises topiramate and a sulfoalkyl ether cyclodextrin of the formula I:

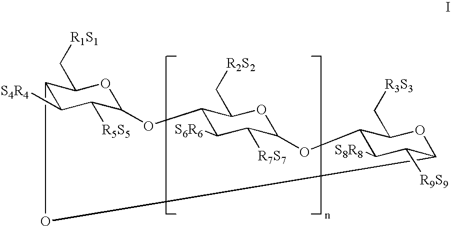

wherein:
n is 4, 5 or 6;
R₁, R₂, R₃, R₄, R₅, R₆, R₇, R₈ and R₉ are each, independently, —O— or a-O—(C₂-C₆ alkylene)-SO₃⁻ group, wherein at least one of R₁ and R₂ is independently a —O—(C₂-C₆ alkylene)-SO₃⁻ group, preferably a —O—(CH₂)ₘSO₃⁻ group, wherein m is 2 to 6, preferably 2 to 4, (e.g., —OCH₂CH₂CH₂SO₃⁻ or —OCH₂CH₂CH₂CH₂SO₃⁻); and
S₁, S₂, S₃, S₄, S₅, S₆, S₇, S₈ and S₉ are each, independently, H or a pharmaceutically acceptable cation which includes, for example, alkali metals (e.g., Li⁺, Na⁺, K⁺), alkaline earth metals (e.g., Ca⁺², Mg⁺²), ammonium ions and amine cations such as the cations of (C₁-C₆)-alkylamines, piperidine, pyrazine, (C₁-C₆)-alkanolamine and (C₄-C₈)-cycloalkanolamine.

In certain embodiments of the invention, the cyclodextrin is a sulfoalkyl ether cyclodextrin derivative described in U.S. Pat. No. 5,134,127 or 5,376,645.

In certain embodiments of the invention, the compositions of the invention are useful for treatment of a condition amenable to treatment with topiramate, which include, e.g., the treatment of epilepsy, seizures (e.g., neonatal seizures), refractory status epilepticus, gambling, migraines, substance dependence, alcoholism; cocaine dependence, nicotine dependence, metabolic syndrome X; diabetes mellitus, type 2, vomiting, obsessive-compulsive disorder, refractory generalized social phobia, Tourette syndrome, levodopa-induced dyskinesia in Parkinson's Disease, refractory POS, Prader-Willi syndrome, multiple sclerosis, Lennox-Gastaut syndrome, bipolar disorder, obesity, post traumatic stress disorder, cluster headaches, severe headaches, anoxia (e.g., neonatal anoxia), and for any condition that can be treated with topiramate (e.g., for patients unable to take oral composition of topiramate).

The compositions of the invention are useful for providing neuroprotection for a patient (e.g., during surgery, e.g., during neonatal or pediatric surgery, e.g., during heart surgery or during a stroke, head injury, or coma).

In certain embodiments, the compositions are useful for protecting brain tissue near an area of ischemic stroke (the penumbra). The compositions may be administered, e.g., within a few hours after a stroke to protect the penumbra brain tissue from injury.

The compositions of the invention are also useful as a counter-measure for chemical warfare nerve agents such as sarin.

The compositions of the invention are also useful as an alternate treatment for a patient, e.g., as a bridge treatment during a period of time when a patient is not able to be treated with an oral formulation of topiramate.

The compositions of the invention are also useful for treating a patient who needs to rapidly attain or re-attain increased plasma topiramate concentrations, e.g., when those concentrations have declined as a result of not taking an oral formulation of topiramate.

The pharmaceutical dosage forms suitable for injection or infusion can include sterile aqueous solutions or dispersions or sterile powders comprising the active ingredient(s) which are adapted for the extemporaneous preparation of sterile injectable or infusible solutions or dispersions. The formulation can be provided as a stock solution, which is diluted with a liquid carrier composition such as dextrose, saline, plasma, or lactated Ringer's solution prior to administration to a patient. The formulation can be provided at a concentration of topiramate that is suitable for administration without dilution. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The formulation can further include a preservative, a solubilizing agent, an antioxidant, a buffering agent, an acidifying agent, a complexation enhancing agent, saline, dextrose, a lyophilizing aid (for example, bulking agents or stabilizing agents), an electrolyte, another therapeutic agent, an alkalizing agent, an antimicrobial agent, an antifungal agent, an antibacterial agent (e.g., a parabens or thimersol) or a combination thereof. Prolonged absorption of the injectable compositions (e.g., by IM injection) can be brought about by the use in the compositions of agents delaying or modifying the absorption, for example, aluminum monostearate, oleaginous vehicles, less soluble salt forms, or poloxamers (block copolymers). The pharmaceutical dosage forms suitable for injection or infusion can include sterile aqueous solutions or dispersions or sterile powders of topiramate which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, sulfoalkyl cyclodextrin in water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants.

Sterile injectable solutions can be prepared by incorporating the active compound(s) into an appropriate solvent with the other optional ingredients enumerated herein, optionally followed by filter sterilization. In the case of sterile powders for the preparation of sterile injectable solutions, the preferred methods of preparation are spray drying, vacuum drying and the freeze drying techniques, which yield a powder of the active ingredient plus any additional desired ingredient present in the previously sterile-filtered solutions.

The concentration of topiramate is, typically, e.g., about 5-100 mg/ml, e.g., 5-50 mg/ml, e.g., 10-20 mg/ml. In some embodiments of the invention, the cyclodextrin, such as a compound of Formula III, is present at a concentration of about 1-700 mg/ml.

As used herein the terms “treat”, “treating” and “treatment” include administering the composition prior to the onset of clinical symptoms of a disease state/condition so as to prevent the development of any symptom, as well as administering the composition after the onset of one or more clinical symptoms of a disease state/condition so as to reduce or eliminate any such symptom, aspect or characteristic of the disease state/condition. Such treating need not be absolute to be useful.

The compositions may, in certain embodiments, be provided in a unit dosage form or in a container from which a dose is measured out. As used herein the term “unit dosage form” relates to a composition containing a specific amount of a drug, the whole of which is intended to be administered as a single dose. It is distinguished from a supply of a multi-dose amount of a medicament, e.g., a bottle of medicine, from which a dose has to be measured out.

As used herein, the term “patient” is taken to mean warm blooded animals such as mammals, for example, cats, dogs, mice, guinea pigs, horses, bovine cows, sheep, and humans.

In certain embodiments of the invention, treatment may include multiple doses, e.g., doses occurring over days, weeks, or years.

The compositions may also include at least one additional therapeutic agent.

In certain embodiments of the invention, the compositions of the invention can be administered to neonatal, pediatric, adult, or geriatric patients. Neonatal patients are of about 0-30 days of age. Pediatric patients are of about up to 17 years of age. Adult patients are at least about 18 years of age. Geriatric patients are at least about 65 years of age.

Topiramate

The compositions of the invention include topiramate (see, e.g., U.S. Pat. Nos. 6,949,518, 6,906,099, 6,699,840, 6,696,091 6,559,293, 6,503,884, 5,952,187, 5,258,402, and 4,513,006). Methods for preparing topiramate are known in the art. Topiramate is designated chemically as 2,3:4,5-Di-O-isopropylidene-β-D-fructopyranosesulfamate and has the following formula II:

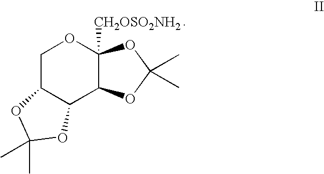

The Cyclodextrin

The compositions of the invention also include a cyclodextrin molecule (e.g., a sulfobutyl ether-β-cyclodextrin such as CAPTISOL®, see, e.g., U.S. Pat. Nos. 6,133,248, 5,874,418, 6,046,177, 5,376,645, 5,134,127, 7,034,013, 6,869,939, WO 2005/117911 and MSDS Number CAP-001). Methods for preparing a sulfobutyl ether-β-cyclodextrin are known in the art. The compositions containing the sulfoalkyl ether cyclodextrin will have improved solubility, stability and/or bioavailability of topiramate.

CAPTISOL® cyclodextrin is a modified cyclodextrin. CAPTISOL® cyclodextrin is a polyanionic beta-cyclodextrin derivative with a sodium sulfonate salt separated from the lipophilic cavity by a butyl ether spacer group, or sulfobutylether (SBE). CAPTISOL® cyclodextrin has been shown to be safe when administered parenterally, orally and via inhalation and does not exhibit the nephrotoxicity associated with beta-cyclodextrin. Relative to beta-cyclodextrin, CAPTISOL® cyclodextrin provides comparable or higher complexation characteristics and superior water solubility in excess of 90 grams/100 ml, a 50-fold improvement. CAPTISOL® cyclodextrin has the following formula III:

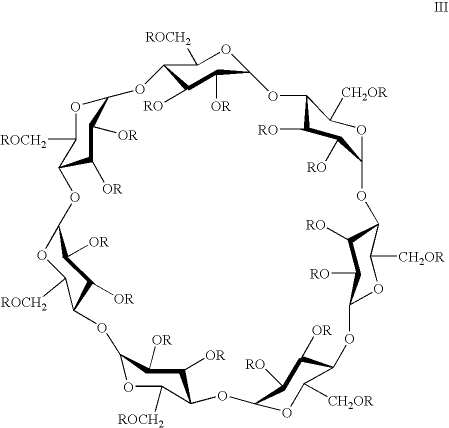

where R═(H)₂₁₋ₓ or (—(CH₂)₄—SO₃Na)ₓ. In certain embodiments, x=6.0-7.1.

In some embodiments of the invention, the cyclodextrin to topiramate mole ratio is about 0.01 to 1.4.

The invention will now be illustrated by the following non-limiting Example.

### EXAMPLE 1

### Topiramate Phase Solubility Study

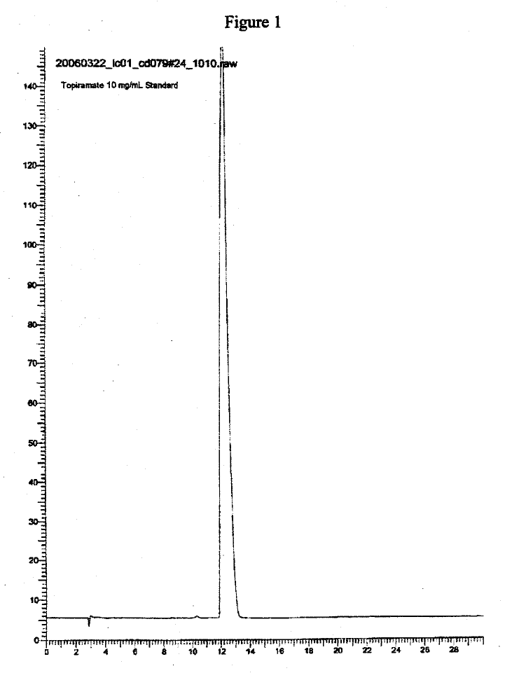

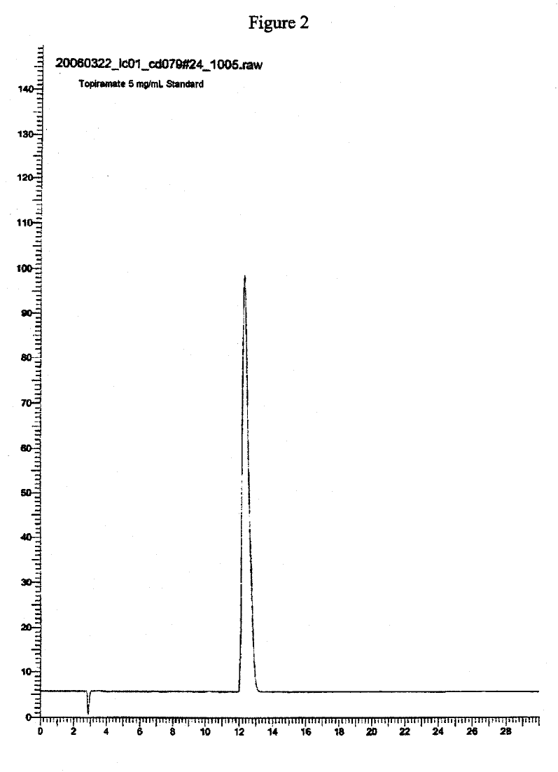

A phase solubility study was conducted with the cyclodextrin CAPTISOL® and topiramate to evaluate the extent of solubilization of the drug by the derivatized cyclodextrin. An HPLC method was modified from the literature and shown to be linear over the range of interest. Chromatograms from injection of two of the topiramate standards are shown in FIGS. 1 and 2.

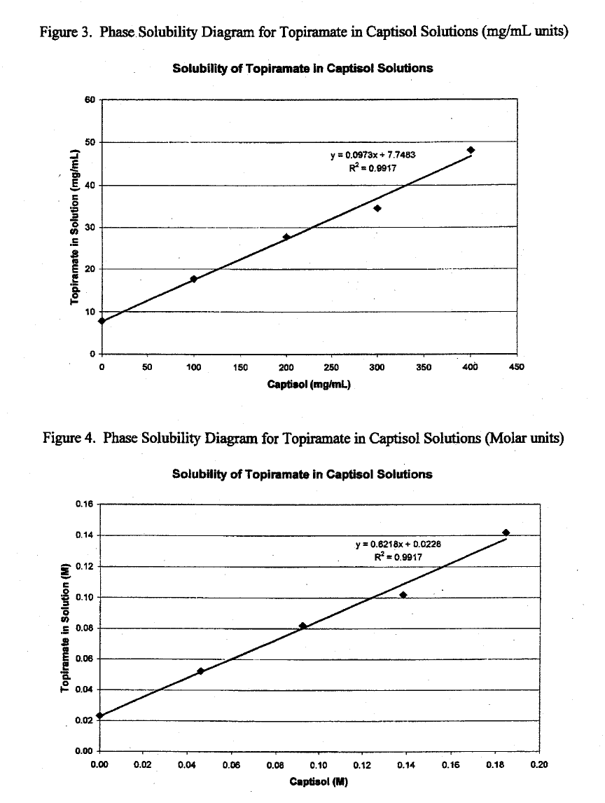

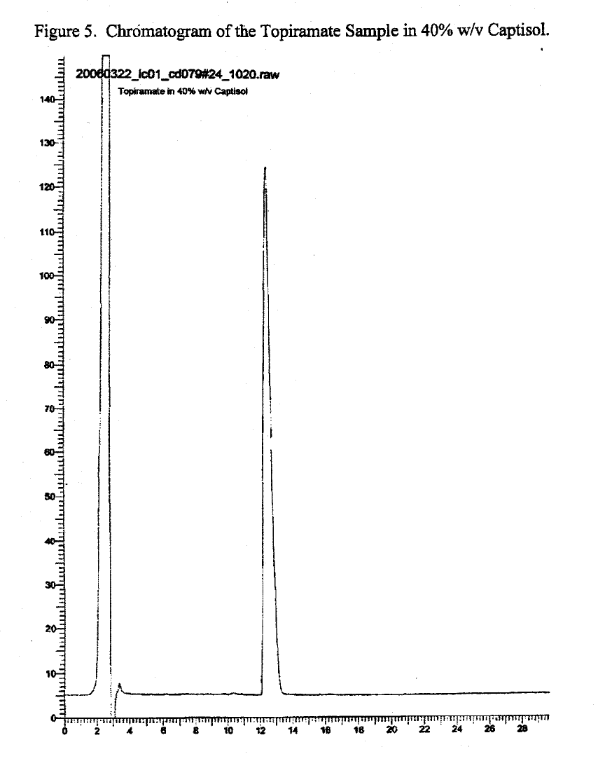

Results of the solubility study are illustrated in FIGS. 3 and 4 and show that topiramate is well solubilized by the cyclodextrin CAPTISOL® in water. Type A-linear phase solubility is observed and a binding constant of 71 M⁻¹ was calculated from the equation: K_{1:1}=slope/S₀(1−slope), where S₀ is the intrinsic solubility of the drug and “slope” is the slope of the molar plot of drug solubility vs cyclodextrin content. The magnitude of the calculated binding constant is low due to the drug being reasonably soluble in water in the absence of cyclodextrin (intrinsic solubility of 7.86 mg/mL). A chromatogram for the analysis of the solubility sample using 40% w/v of the cyclodextrin CAPTISOL® is given in FIG. 5.

### Methods:

Solutions containing increasing amounts of dissolved CAPTISOL® brand of sulfobutylether b-cyclodextrin were prepared and added to small glass vials. Excess solid topiramate was added to each vial and the vials were capped, vortexed and placed in constant agitation for five days at room temperature (˜23-25° C.). If any vial showed complete dissolution of the added drug, additional drug was added and the vial returned to the stirring mode.

After the multiday equilibration period, the vials were centrifuged (twice at 693×g, 25° C.) and aliquots were taken from the clear supernatant solutions. The aliquots were diluted 1:3 (1:5.67 for 40% cyclodextrin CAPTISOL® solutions) with mobile phase and analyzed by HPLC for topiramate content.

### Materials:

Topiramate: Lot #LL-001-009-III-01 (Divi's Laboratories Ltd, Ameerpet, Hyderabad 500016, India)

The cyclodextrin CAPTISOL®: Lot #17CX01.HQ00009 (CyDex, Inc. Lenexa, Kans.)

### Chromatography:

### Chromatographic Conditions

| | |
|---|---|
| HPLC: | Dionex |
| Detection: | Refractive Index Detector |
| Column: | SB-Phenyl (5 μm) 250 mm × 4.6 mm |
| Column Temperature: | 35° C. |
| Mobile Phase: | 40:60 MeOH:Water |
| Flow Rate: | 1.0 mL/min isocratic |
| Run Time: | 30 minutes |
| Sample Solvent: | Mobile phase |
| Injection Volume: | 50 μL |
| Retention Time: | ~12.5 minutes |

### Mobile Phase Preparation

Combined 400 mL of methanol and 600 mL of water, mixed well and filtered.

### Standard Solutions

1—TPM10 Solution: ˜250 mg of TPM was weighed into a 25 mL volumetric flask, diluted to volume with mobile phase, and mixed well.

2—TPM5 Solution: 5 mL of TPM10 solution were transferred into a 10 mL volumetric flask and diluted to volume with mobile phase.
3—TPM1 Solution: 1 mL of TPM10 solution was transferred into a 10 mL volumetric flask and diluted to volume with mobile phase.

All publications, patents and patent applications cited herein are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

What is claimed is:

1. A composition comprising topiramate, or a salt thereof, and compound of Formula I: 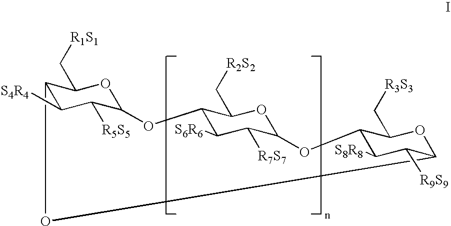
wherein:
n is 4, 5 or 6;
R₁, R₂, R₃, R₄, R₅, R₆, R₇, R₈ and R₉ are each, independently, —O— or a —O—(C₂-C₆ alkylene)-SO₃⁻ group, wherein at least one of R₁ and R₂ is independently a —O—(C₂-C₆ alkylene)-SO₃⁻ group; and
S₁, S₂, S₃, S₄, S₅, S₆, S₇, S₈ and S₉ are each, independently, H or a pharmaceutically acceptable cation.

2. The composition of claim 1, wherein at least one of R₁ and R₂ is independently a —O—(C₂-C₆ alkylene)-SO₃⁻ group that is a —O—(CH₂)ₘSO₃⁻ group, wherein m is 2 to 6, and the pharmaceutically acceptable cation is H, an alkali metal, an alkaline earth metal, an ammonium ion, or an amine cation.

3. The composition of claim 1, wherein the compound of Formula I is a compound of Formula III: 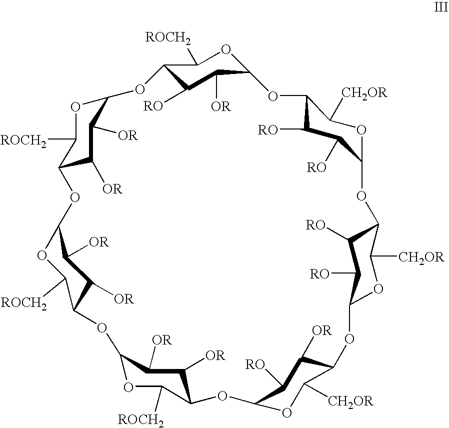
wherein R═(H)₂₁₋ₓ or (—(CH₂)₄—SO₃Na)ₓ.

4. The composition of claim 1, wherein the composition further comprises an additional therapeutic agent.

5. The composition of claim 1, wherein the topiramate is present in the composition at a concentration of about 5-100 mg/ml.

6. The composition of claim 5, wherein the topiramate is present in the composition at a concentration of about 5-50 mg/ml.

7. The composition of claim 6, wherein the topiramate is present in the composition at a concentration of about 10-20 mg/ml.

8. The composition of claim 1, wherein the compound of Formula I is present in the composition at a concentration of about 1-700 mg/ml.

9. The composition of claim 1, wherein the composition further comprises a pharmaceutically-acceptable carrier.

10. The composition of claim 1, wherein the composition is suitable for injectable administration to a patient.

11. The composition of claim 10, wherein the composition is suitable for intravenous administration to a patient.

12. The composition of claim 2, wherein x=6.0-7.1.

13. A method for delivering topiramate to a patient, comprising administering the composition of claim 1 to the patient.

14. A method for treating a patient who has or is at risk for developing a condition amenable to treatment with topiramate comprising administering an effective amount of the composition of claim 1 intravenously to the patient so as to treat the condition.

15. The method of claim 14, wherein the condition is selected from epilepsy, status epilepticus, refractory status epilepticus, gambling addiction, migraines, substance dependence, alcoholism; cocaine dependence, nicotine dependence, metabolic syndrome X; diabetes mellitus, type 2, vomiting, obsessive-compulsive disorder, refractory generalized social phobia, Tourette syndrome, levodopa-induced dyskinesia in Parkinson's Disease, refractory POS, Prader-Willi syndrome, multiple sclerosis, Lennox-Gastaut syndrome, bipolar disorder, obesity, post traumatic stress disorder, cluster headaches, severe headaches, and conditions caused by exposure to a chemical warfare nerve agent.

16. A method for providing neuroprotection in a patient, comprising administering an effective amount of the composition of claim 1 intravenously to the patient.

17. The method of claim 16, wherein the neuroprotection is needed during surgery.

18. A method for treating anoxia in a patient, comprising administering an effective amount of the composition of claim 1 intravenously to the patient.

19. A method for treating seizures in a patient, comprising administering an effective amount of the composition of claim 1 intravenously to the patient.

20. A method for treating a stroke in a patient, comprising administering an effective amount of the composition of claim 1 intravenously to the patient.

21. A method for loading a patient to attain an effective topiramate concentration, comprising administering an effective amount of the composition of claim 1 intravenously to the patient.

22. The method of claim 13, wherein oral topiramate therapy for the patient has been interrupted.

23. The method of claim 13, wherein the patient is a neonatal patient.

24. The method of claim 13, wherein the patient is a pediatric patient.

25. The method of claim 13, wherein the patient is an adult patient.

26. The method of claim 13, wherein the patient is a geriatric patient.
